(19) United States
(12) Patent Application Publication
(10) Pub. No.: US 2024/0177828 A1
(43) Pub. Date: May 30, 2024

(54) INFORMATION PROCESSING SYSTEM, INFORMATION PROCESSING DEVICE, INFORMATION PROCESSING METHOD, AND NON-TRANSITORY COMPUTER-READABLE MEDIUM STORING PROGRAM

(71) Applicant: NEC Corporation, Minato-ku, Tokyo (JP)

(72) Inventors: Koichi Nihei, Tokyo (JP); Yoshifumi Onishi, Tokyo (JP); Takanori Iwai, Tokyo (JP); Koichi Kawashima, Tokyo (JP); Akira Yamauchi, Tokyo (JP)

(73) Assignee: NEC Corporation, Minato-ku, Tokyo (JP)

(21) Appl. No.: 18/275,144

(22) PCT Filed: Sep. 29, 2021
(86) PCT No.: PCT/JP2021/035823

§ 371 Date: Jul. 31, 2023

(51) Int. Cl. G16H 30/20; G06T 7/00; G06T 7/20; G06V 10/25; G16H 10/60; G16H 30/40

(52) U.S. Cl. CPC G16H 30/20; G06T 7/0002; G06T 7/20; G06V 10/25; G16H 10/60; G16H 30/40; G06T 2207/30168; G06T 2207/30196

(57) ABSTRACT

An information processing system (100) according to the present disclosure includes: an acquisition means (101) for acquiring video data including a subject receiving a medical service and subject-related information related to the subject included in the video data; an identification means (102) a change area where image quality in the video data is changed based on the subject-related information; and an image quality changing means (103) for changing the image quality in the identified change area. The subject-related information is at least one of the subject's medical record information, medical interview information, subject's voice information, or motion information included in the video data.

### TECHNICAL FIELD

The present disclosure relates to an information processing system, an information processing device, an information processing method, and a non-transitory computer-readable medium storing a program.

### BACKGROUND ART

The introduction of medical service delivery systems that transmit video data from remote locations via networks to providers of medical services is expanding.

The following is a patent literature on image quality.

Patent Literature 1 discloses a technique for analyzing high frequency components in each area of the acquired video and adjusting the image quality so that at least some of the areas of the video have higher quality as the high-frequency components increases, according to the analysis results. This technology is said to be capable of reducing the amount of video data and making the difference in image quality at the boundary between the low image quality area and high image quality area less noticeable.

In addition, Patent Literature 1 describes changing the image quality setting in the image quality level map, which is generated by setting the image quality for each area based on the analysis results of high-frequency components, so that when the face area of a person is detected, the image quality of the face area is higher than that of the areas other than the face area.

### CITATION LIST

### Patent Literature

Patent Literature 1: Japanese Unexamined Patent Application Publication No. 2020-058014

### SUMMARY OF INVENTION

### Technical Problem

Patent Literature 1 only discloses detecting a face area by extracting facial feature points and making the image quality of the face area higher than that of other areas, and does not disclose a technique for appropriately controlling the image quality of necessary areas in a medical service delivery system that transmits video data to a provider of medical services from a remote location via a network.

An object of the present disclosure is to provide an information processing system, an information processing device, an information processing method, and a non-transitory computer-readable medium storing a program, which can control image quality in the area appropriately necessary for a provider of medical services.

### Solution to Problem

An information processing system according to an aspect includes: an acquisition means for acquiring video data including a subject receiving a medical service and subject-related information related to the subject included in the video data; an identification means for identifying a change area where image quality in the video data is changed based on the subject-related information; and an image quality changing means for changing the image quality in the identified change area.

An information processing device according to an aspect includes: an acquisition means for acquiring video data including a subject receiving a medical service and subject-related information related to the subject included in the video data; an identification means for identifying a change area where image quality in the video data is changed based on the subject-related information; and a control means for outputting an image quality change instruction to change the image quality in the identified change area.

An information processing method according to an aspect includes: acquiring video data including a subject receiving a medical service and subject-related information related to the subject included in the video data; identifying a change area where image quality in the video data is changed based on the subject-related information; and changing the image quality in the identified change area.

A non-transitory computer-readable medium according to an aspect stores a program for causing a computer to execute: processing of acquiring video data including a subject receiving a medical service and subject-related information related to the subject included in the video data; processing of identifying a change area where image quality in the video data is changed based on the subject-related information; and processing of changing the image quality in the identified change area.

### Advantageous Effects of Invention

According to the above aspect, it is possible to provide an information processing system, an information processing device, an information processing method, and a non-transitory computer-readable medium storing a program, which can clarify videos of areas necessary for a provider of medical services.

### BRIEF DESCRIPTION OF DRAWINGS

FIG. 1 is a block diagram illustrating a configuration of an information processing system according to an example embodiment.

FIG. 2 is a diagram illustrating a configuration example of an information processing system including an information processing device according to an example embodiment.

FIG. 3 is a block diagram illustrating an example of a configuration of the doctor-side terminal in FIG. 2.

FIG. 4 is a block diagram illustrating an example of a configuration of the patient-side terminal in FIG. 2.

FIG. 5 is a diagram illustrating an example of the display contents of the doctor-side terminal.

FIG. 6 is a diagram illustrating an example of the display contents of the patient-side terminal.

FIG. 7 is a sequence diagram illustrating an example of the flow of information processing according to the example embodiment.

### EXAMPLE EMBODIMENT

Example embodiments of the present disclosure are described below with reference to the drawings. Note that in the description and drawings described below, omission and simplification are made as appropriate, for clarity of description. Furthermore, in each of the drawings described below, the same elements are denoted by the same reference signs, and a duplicate description is omitted as necessary. In addition, specific numerical values and the like shown below are only examples to facilitate understanding of the present disclosure, and are not limited thereto.

The example embodiment relates to a technology for providing medical services, in which video data of a subject is transmitted via a network between a facility providing medical services and a remote facility with a subject receiving medical services, and a doctor or other specialist makes a diagnosis based on the subject's video.

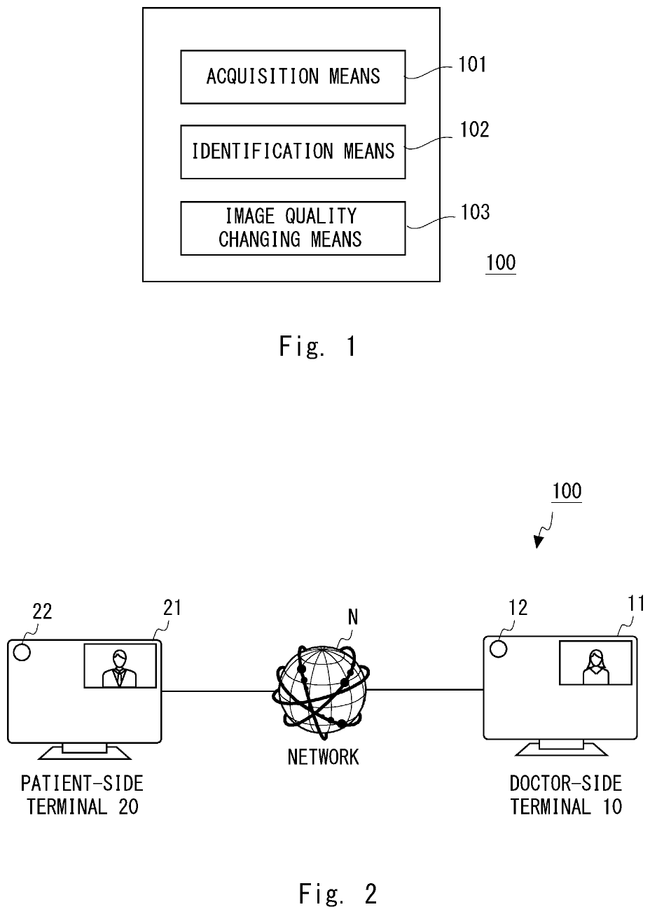

FIG. 1 is a block diagram illustrating a configuration of an information processing system 100 according to an example embodiment. As illustrated in FIG. 1, the information processing system 100 includes an acquisition means 101, an identification means 102, and an image quality changing means 103. The acquisition means 101 acquires video data including a subject receiving a medical service and subject-related information related to the subject included in the video data. The identification means 102 identifies the change area to change the image quality in the video data based on the subject-related information. The image quality changing means 103 changes the image quality in the specified change area.

In this way, by identifying the area whose image quality is to be changed in the video data based on the subject-related information of the subject, it is possible to change the image quality in the areas necessary for the doctor to make a diagnosis, to clarify the video, and to improve medical services.

Next, a specific example of the information processing system 100 according to the example embodiment will be described with reference to FIGS. 2 to 4. FIG. 2 is a diagram illustrating a configuration example of the information processing system 100 including an information processing device 1 according to the example embodiment. As illustrated in FIG. 2, the information processing system 100 includes a doctor-side terminal 10 and a patient-side terminal 20.

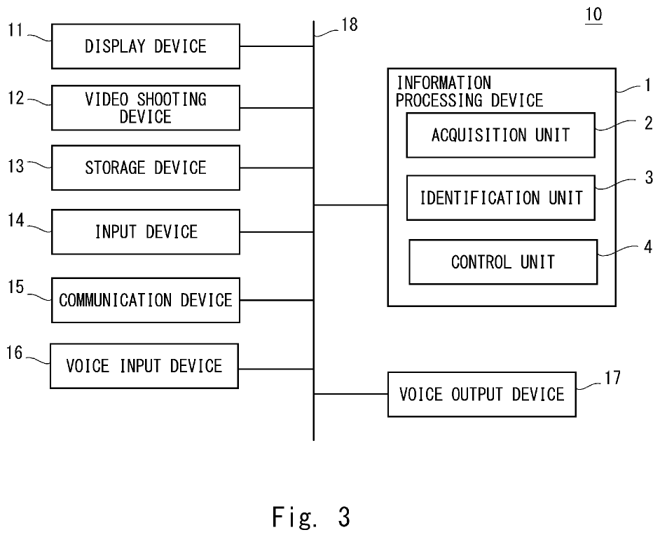

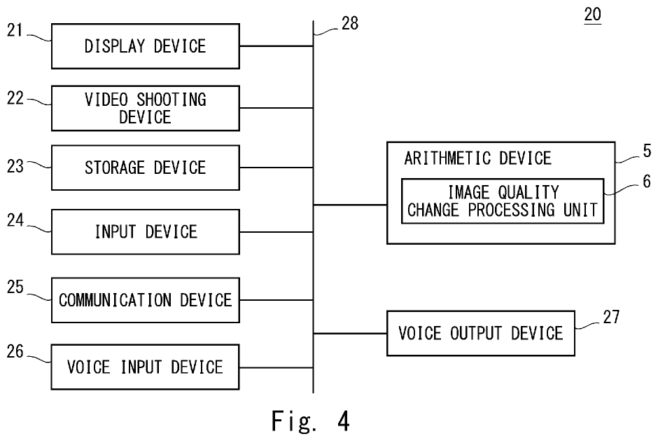

FIG. 3 is a block diagram illustrating an example of the doctor-side terminal 10 in FIG. 2. FIG. 3 illustrates the hardware configuration of the doctor-side terminal 10 as well as the logical functional blocks realized in the information processing device 1 to execute the image quality change operation. FIG. 4 is a block diagram illustrating an example of the patient-side terminal 20 in FIG. 2. FIG. 4, illustrates the hardware configuration of the patient-side terminal 20 as well as the logical functional blocks realized in the arithmetic device 5 to execute the image quality change operation.

The information processing system 100 supports telemedicine (on-line medical care and home medical care), in which a subject receiving medical services receives various services from a medical service provider while staying at home. Here, medical service providers include doctors, nurses, physical therapists, and care workers and the like. Telemedicine also includes medical treatment in the narrow sense, such as treatment and procedures performed by doctors and the like, as well as medical treatment in the broad sense, such as rehabilitation performed by physical therapists, health management and medication administration by care workers. Hereinafter, in order to simplify the description, the medical service provider is referred to as a “doctor”.

Examples of subjects of medical services include patients, persons receiving medical examinations (medical examinees), and persons receiving nursing care (care receivers). Hereinafter, in order to simplify the description, the subject of medical services is referred to as a “patient”. Facilities that provide medical services are, for example, hospitals, clinics, and nursing care facilities. Hereinafter, in order to simplify the description, the facility that provides medical services is referred to as a “hospital”.

The doctor-side terminal 10 and the patient-side terminal 20 are connected by a network N and communicate bidirectionally in real time. Here, the network N is a communication network such as the Internet or a dedicated line. In the case of the Internet, it is preferable that a closed network is formed by a virtual private network (VPN) or the like.

The doctor-side terminal 10 is installed at a hospital, and the patient-side terminal 20 is installed at a patient's home in a remote location. The doctor-side terminal 10 and the patient-side terminal 20 may be a personal computer, a server machine, a tablet terminal with touch panel input/output, a smartphone, or the like. A doctor in a hospital can use the doctor-side terminal 10 to communicate in real time with a patient using the patient-side terminal 20 in a remote location.

Such bidirectional communication of video data including voice and motion of the doctor and the patient imposes a high network load, so there is a need to reduce the size of the video data. In general, video data is compressed and encoded to reduce the bandwidth required for transmission. However, when video data is compressed and encoded, image quality is deteriorated.

In telemedicine, a doctor makes a diagnosis based on video transmitted from the patient's home. Therefore, if the video deteriorates, some abnormal parts in the image, such as the injured part of the patient, may become unclear, which may lead to misdiagnosis. The information processing device 1 according to the example embodiment identifies abnormal parts that vary from patient to patient using the subject-related information described later, and makes such areas clearer by making the image quality of the identified areas higher than that of other areas.

As illustrated in FIG. 3, the doctor-side terminal 10 includes an information processing device 1, a display device 11, a video shooting device 12, a storage device 13, an input device 14, a communication device 15, a voice input device 16, and a voice output device 17, which are interconnected by a bus 18. The information processing device 1 is an arithmetic processing device such as a central processing unit (CPU) or a graphics processing unit (GPU). The information processing device 1 includes an acquisition unit 2, an identification unit 3, and a control unit 4 as logical functional blocks. The acquisition unit 2, the identification unit 3, and the control unit 4 correspond to the acquisition means, the identification means, and the control means described in the claims.

The acquisition unit 2 acquires video data including a subject and subject-related information related to the subject included in the video data. The video data including the patient is transmitted from the patient-side terminal 20 via the network, for example. The video data including the patient may be stored in a database (not illustrated) connected to the network. In this case, the acquisition unit 2 can access the database to acquire the video data.

The subject-related information includes the patient's personal information, medical record information, medical interview information, biometric information, ability information indicating the patient's ability to perform activities of daily living such as activities of daily living (ADL), and records of prescriptions, care, and rehabilitation and the like. These pieces of information are stored in the storage device 13. Note that these pieces of information may be stored and managed in an external management server.

The subject-related information also includes the patient's voice information and motion information included in the patient's video data transmitted from the patient-side terminal 20, which will be described later.

The identification unit 3 identifies the change area to change the image quality in the video data based on the subject-related information. The identification unit 3 can identify the change area by combining one or more of the plurality of pieces of subject-related information described above. The change area is an area in the video containing the patient that the doctor should visually examine in order to make a diagnosis.

For example, the identification unit 3 can identify the injured part of the patient from the description of past medical information in the medical record and identify the abnormal area corresponding to the injured part in the video as the change area. Note that, the area to be identified as the change area may be changed depending on the first visit, second visit, number of medical examinations, degree of symptoms, and the like.

It is also possible to identify a change area based on the contents of the medical questionnaire that the patient has completed before the medical examination. Furthermore, based on the patient's voice information included in the patient's video data, the identification unit 3 can identify as a change area the part where the patient complains of abnormality by voice, which is not included in the medical record information or the medical interview information.

It is also possible to identify the change area based on the patient's motion information included in the patient's video data. For example, in a case where the patient is spasming, the acquisition unit 2 acquires the minute vibrations caused by the patient's spasms in the video data as motion information. Based on this motion information, the identification unit 3 can identify the part of spasm as the change area.

Furthermore, in a case where the patient's voice information and motion information are acquired, the identification unit 3 can identify the change area based on both the voice information and the motion information. For example, the identification unit 3 can identify the part in the video where the patient is pointing his/her finger as the change area based on the voice information of the patient saying “I have pain here” and the motion information of the patient pointing his/her finger.

Furthermore, the identification unit 3 can identify the change area according to the doctor's input operation. For example, an input means that imitates the human body (hereinafter referred to as a “human body model input device”), including three-dimensional human body models such as human models and dolls, and two-dimensional human body models such as illustrations of the human body, can be used as input devices that accept doctor's input operations.

When the doctor presses the part to be visually examined on the human body model input device, specified position information is generated to specify the change area in the patient's video data. The identification unit 3 can identify the change area based on the specified position information. Therefore, the above-described subject-related information also includes specified position information by the human body model input device.

When the area identified as a change area is not included in the medical record information or medical interview information as an abnormal part of the patient, the identification unit 3 can automatically add information about the part to the medical record or the like. For example, in a case where a change area is specified based on a conversation between a doctor and a patient (voice information), the change area is added to the medical record as an abnormal part. It is also possible to add symptoms and other information about the added abnormal part to the medical record by analyzing the doctor's voice information.

The control unit 4 outputs an image quality change instruction to the patient-side terminal 20 to change the image quality in the identified change area. The control unit 4 outputs an image quality change instruction to make the image quality in the change area higher than that of the areas other than the change area.

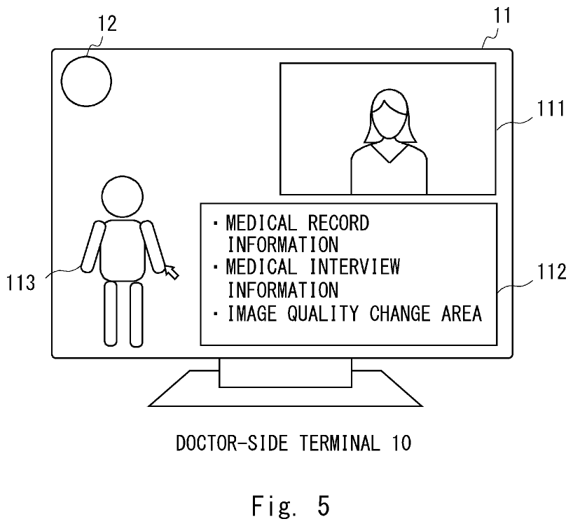

The display device 11 is a liquid crystal display (LCD) or the like, and displays various types of information. FIG. 5 is a diagram illustrating an example of display contents of the doctor-side terminal. As illustrated in FIG. 5, the display device 11 includes a video display area 111, an information display area 112, and an input information display area 113.

In the video display area 111, the patient's video transmitted from the patient-side terminal 20 is displayed. As described above, in a case where the image quality change instruction is output from the control unit 4 to the patient-side terminal 20, the patient-side terminal 20 changes the image quality in the video data so that the change area has a higher image quality than the areas other than the change area when transmitting the video data including the patient to the doctor-side terminal 10.

For example, in a case where the patient-side terminal 20 performs compression coding to reduce the data size when transmitting video data, by reducing the compression ratio of the change area compared to that of the areas other than the change area, the video data can be of higher image quality in the change area than in the areas other than the change area. As a result, areas necessary for diagnosis by the doctor can be displayed with higher image quality than other areas, and the influence of image quality deterioration on medical services can be suppressed.

The information display area 112 displays medical record information (for example, chronic diseases, medical history, examination information, and the like) and medical interview information. In the information display area 112, information indicating the image quality change area, which is identified based on the subject-related information and in which the image quality has been changed, is displayed. Note that the doctor-side terminal 10 may further include a voice analysis unit, and the information display area 112 may display information related to the content of the voice recognition of the conversation between the doctor and the patient. For example, predetermined vital signs (blood pressure, heart rate, or the like) can be displayed based on voice information of the patient saying “I have a headache”.

Furthermore, the information display area 112 may display candidate disease names according to symptoms by utilizing the voice recognition results of the conversation between the doctor and the patient. In addition, after narrowing down the disease name from the symptoms, the information display area 112 may display recommendation information on what questions to ask in order to determine the disease name.

In the input information display area 113, a diagram imitating the human body is displayed. When the specified position information is generated by the human body model input device described above, marks, icons, and others displayed on the part corresponding to the specified position information in the diagram of the human body. That is, the input information display area 113 displays a clear indication of the patient's body part to be visually examined, which the doctor presses down on the human body model input device.

The video shooting device 12 is, for example, a video camera, and can image the doctor using the doctor-side terminal 10. The video shooting device 12 shoots the doctor in a predetermined shooting range (shooting angle of view) at a predetermined shooting rate to generate video data including a plurality of pieces of video data in a time series.

The storage device 13 is, for example, a non-volatile storage device such as a hard disk drive (HDD), a solid state drive (SSD), or a flash memory. The storage device 13 stores a program for realizing each function of the components included in the information processing device 1. When the information processing device 1 executes this program, each function of the components included in the information processing device 1 is realized.

The storage device 13 may include a memory such as a random access memory (RAM) or a read only memory (ROM). When executing the program, the information processing device 1 may execute the program after reading the program on the memory, or may execute the program without reading the program on the memory. The storage device 13 also plays a role of storing information (subject-related information and the like) and data held by the components included in the information processing device 1.

The input device 14 is a device that accepts operational input from the doctor who is the user of the doctor-side terminal 10, such as a keyboard and a mouse. The display device 11 and the input device 14 may be integrated to be realized as a touch panel. The input device 14 also includes the human body model input device described above.

The communication device 15 transmits and receives data to and from the external patient-side terminal 20 via, for example, a wireless transmission channel. For example, the communication device 15 transmits video data including the doctor to the patient-side terminal 20, and receives video data including the patient from the patient-side terminal 20. In addition, in a case where the medical record information, the medical interview information, and the like are stored in an external management server, the communication device 15 receives these pieces of information from the management server.

The voice input device 16 is, for example, a microphone, and collects the voice uttered by the doctor as voice data. Video data is generated by combining video data generated by the video shooting device 12 and voice data collected by the voice input device 16. That is, the video data includes the doctor's motion information and voice data. The voice output device 17 is, for example, a speaker, and reproduces the voice included in the patient's video data transmitted from the patient-side terminal 20.

The acquisition unit 2 may further acquire voice data generated by the doctor that is collected by the voice input device 16. The identification unit 3 can identify the change area based on at least one of the subject-related information and the doctor's voice data. Furthermore, in a case where the voice information included in the subject's video data and the doctor's voice data are acquired, the identification unit 3 may give priority to the doctor's voice data and identify the change area based on the voice data.

For example, in a case where the patient says, “I have a pain in my hand,” and then the doctor checks the patient's video image and says, “You have pain in the first joint of the index finger of your right hand,” the identification unit 3 can identify “the first joint of the index finger of the right hand” as the change area based on the doctor's voice data. As a result, areas necessary for diagnosis by the doctor can be converted to high image quality.

In addition, the identification unit 3 can function as an output means for outputting a plurality of candidate change areas. For example, the identification unit 3 can output the first change area identified using the medical record information and the second change area, which is different from the first change area, identified using the medical interview information, as candidate change areas. These candidates can be displayed in the information display area 112.

The doctor can select at least one candidate from a plurality of candidate change areas using the input device 14. That is, the input device 14 functions as an accepting means for accepting the selection of candidates. The control unit 4 outputs an image quality change instruction to change the image quality of the area corresponding to the selected candidate. As a result, the doctor himself/herself can appropriately select the candidate that he/she considers necessary for visual examination from the plurality of candidate change areas automatically presented to him.

Note that the plurality of candidate change areas may be narrowed down according to the diagnosis and treatment department in which the patient is being examined. That is, a predetermined candidate is set for each clinical department, and candidate change areas that are not included in this predetermined candidate can be excluded. For example, in a case where the clinical department is “ophthalmology”, the candidates for ophthalmology shall not include the “fingertips of the hand” of the patient's body part. When a patient is undergoing an online ophthalmology consultation and “fingertips of the hand” is identified as a change area candidate, the candidate change area indicating “fingertips of the hand” that is not included in the candidates for ophthalmology can be excluded.

In addition, when a plurality of candidate change areas are displayed in the information display area 112, they may be displayed in a priority order. For example, based on the medical record information, the candidate change area with severe symptoms can be displayed as the highest priority. In this case, the contents of the medical record and the candidate change area (abnormal part of the patient) may be tied together and displayed in the information display area 112 so that the doctor knows why the candidate change area is the highest priority.

Next, the patient-side terminal 20 will be described. As illustrated in FIG. 4, the patient-side terminal 20 includes an arithmetic device 5, a display device 21, a video shooting device 22, a storage device 23, an input device 24, a communication device 25, a voice input device 26, and a voice output device 27, which are mutually connected by a bus 28.

Note that the display device 21, the video shooting device 22, the storage device 23, the input device 24, the communication device 25, the voice input device 26, and the voice output device 27 have the same general functions as those of the display device 11, the video shooting device 12, the storage device 13, the input device 14, the communication device 15, the voice input device 16, and the voice output device 17, respectively, and thus, description thereof is appropriately omitted.

The arithmetic device 5 is, for example, an arithmetic processing device such as a CPU or a GPU. The arithmetic device 5 includes an image quality change processing unit 6 as a logical functional block. The image quality change processing unit 6 changes the image quality of the video data including the patient according to the image quality change instruction from the doctor-side terminal 10.

For example, in a case the image quality change processing unit 6 has a codec unit that performs compression coding when transmitting video data containing patients, it changes the compression ratio of the video data in the codec unit. When an image quality change instruction is received from the doctor-side terminal 10, the compression ratio of the change area is reduced below that of the areas other than the change area, thereby generating video data in which the change area has higher image quality than the areas other than the change area. As a result, the overall data size of the video data to be transmitted can be reduced while making the change area high image quality.

Note that, the codec unit may set two compression ratios: a low compression ratio (high-resolution image quality), which is the compression ratio of the change area, and a high compression ratio (low-resolution image quality), which is the compression ratio of the areas other than the change area. Furthermore, the compression ratio may be set at three or more levels, for example, the change area, the area including the patient, and the background area.

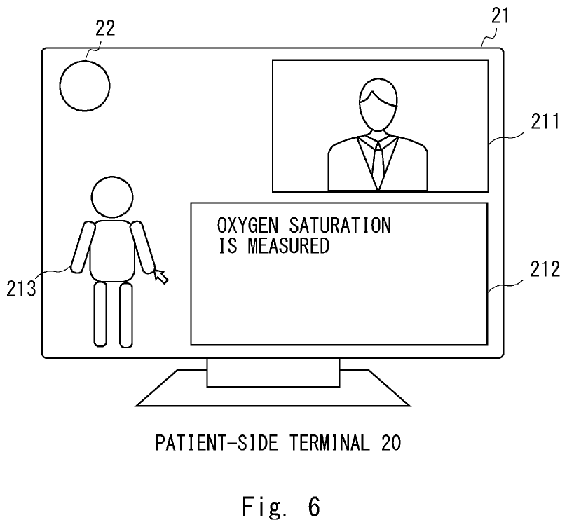

The display device 21 is an LCD or the like, and displays various types of information. FIG. 6 is a diagram illustrating an example of the display contents of the patient-side terminal. The display device 21 includes a video display area 211, an information display area 212, and an input information display area 213. In the video display area 211, an image including the doctor transmitted from the doctor-side terminal 10 is displayed.

In the information display area 212, notification information to the patient is displayed. For example, in a case where it is necessary to measure oxygen saturation based on the patient's video based on subject-related information, a display notifying the patient that oxygen saturation measurement is to be performed can be provided in the information display area 212. From this notification, the patient knows what is being measured and can avoid moving his or her own body part as much as possible, which is necessary for the measurement.

In the input information display area 213, a diagram imitating the human body is displayed. Similar to the input information display area 113, when specified position information is generated by the human body model input device described above, marks, icons, and others are displayed on the part corresponding to the specified position information in the diagram of the human body. That is, the input information display area 213 clearly displays the part of the patient to be visually examined, where the doctor pressed the human body model input device. From this display, the patient knows which part of his or her body is being visually examined by the doctor and can avoid moving that part of the body.

As described above, the information display area 212 and the input information display area 213 function as a notification means to notify the patient of the change area (the patient's body part to be visually examined by the doctor) where the image quality is to be changed. The display device 21 is described as an example of a notification means to display various indications, but the notification means is not limited to this. For example, the patient may be notified of the change area by voice.

The video shooting device 22 is, for example, a video camera, and can image the patient using the patient-side terminal 20. The video shooting device 22 shoots the patient in a predetermined shooting range (shooting angle of view) at a predetermined shooting rate to generate video data including a plurality of pieces of video data in a time series.

The storage device 23 is a non-volatile storage device such as an HDD, an SSD, or a flash memory. The storage device 23 stores a program for realizing the function of the image quality change processing unit 6 included in the arithmetic device 5. When the arithmetic device 5 executes this program, the functions of the image quality change processing unit 6 are realized. Similar to the storage device 13, the storage device 23 may include a memory such as a RAM or a ROM. The storage device 13 also plays a role of storing data and the like necessary for the image quality change processing unit 6 to perform processing of changing the image quality of the video data.

The input device 24 is a device that accepts operational input from the patient who is the user of the patient-side terminal 20, such as a keyboard and a mouse. The display device 21 and the input device 24 may be integrated to be realized as a touch panel.

The communication device 25 transmits and receives data to and from the external doctor-side terminal 10 via, for example, a wireless transmission channel. For example, video data including the doctor and video data including the patient are transmitted via the communication device 25. Furthermore, in a case where the image quality change instruction is received, the communication device 25 transmits, to the doctor-side terminal 10, video data in which the image quality in the change area of the video data including the patient is higher than that of the video data other than the change area.

The voice input device 26 is, for example, a microphone, and collects the voice uttered by the patient. Video data is generated by combining video data generated by the video shooting device 22 and voice information collected by the voice input device 26. That is, the video data includes the patient's motion information and voice information. The voice output device 27 is, for example, a speaker, and reproduces the voice included in the patient's video data transmitted from the doctor-side terminal 10.

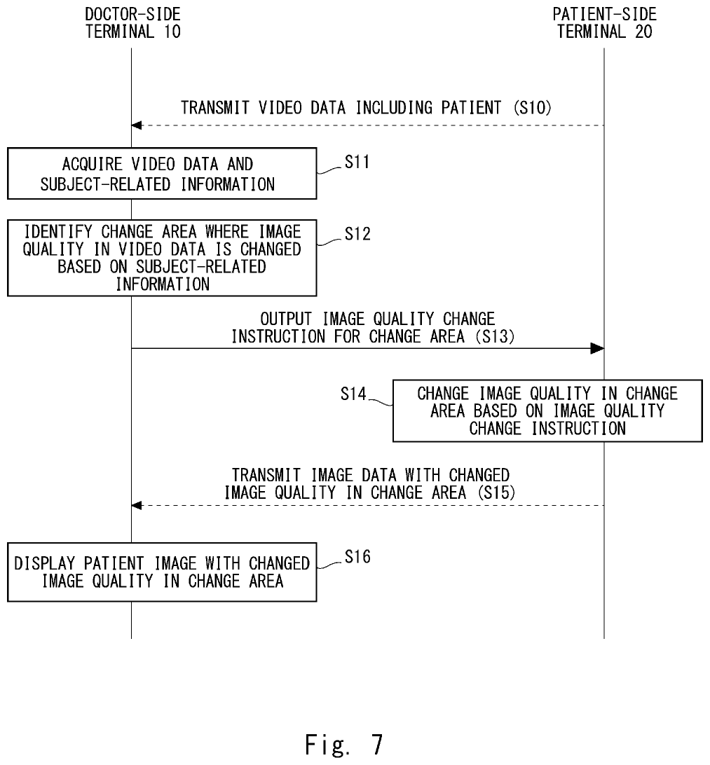

Here, an information processing method according to the example embodiment will be described with reference to FIG. 7. FIG. 7 is a sequence diagram illustrating an example of the flow of information processing according to the example embodiment. First, the patient-side terminal 20 transmits video data including the patient to the doctor-side terminal 10 (S10). The acquisition unit 2 of the doctor-side terminal 10 acquires the video data including the patient and the subject-related information of the patient (S11). Then, the identification unit 3 identifies the change area to change the image quality in the video data based on the subject-related information (S12).

Then, the control unit 4 generates an image quality change instruction to change the image quality in the identified change area and outputs it to the patient-side terminal 20 via the communication device 15 (S13). Specifically, the control unit 4 outputs an image quality change instruction to make the image quality in the change area higher than the image quality in the areas other than the change area. For example, when transmitting the video data generated by the video shooting device 22 of the patient-side terminal 20 to the doctor-side terminal 10, the control unit 4 can generate an image quality change instruction to lower the compression ratio of the change area compared to the compression ratio of the areas other than the change area to make the change area high image quality.

The image quality change processing unit 6 of the patient-side terminal 20 changes the image quality in the identified change area in the video data including the patient based on the image quality change instruction (S15). More specifically, the image quality change processing unit 6 generates video data in which the image quality in the change area is higher than the image quality in the areas other than the change area in the video data including the patient. When transmitting the video data generated by the video shooting device 22 to the doctor-side terminal 10, the image quality change processing unit 6 lowers the compression ratio of the change area compared to the compression ratio of the areas other than the change area to make the change area high image quality.

The generated video data is transmitted to the doctor-side terminal 10 via the communication device 25 (step S16). The display device 11 of the doctor-side terminal 10 displays an image in which the image quality in the change area is higher than the image quality in the areas other than the change area (step S17). As a result, the video can be automatically made clearer in the change area, that is, the area the doctor needs to make a diagnosis.

As described above, according to the example embodiment, it is possible to identify the area to be visually examined from subject-related information, which is information necessary for medical examination, such as past medical information in medical records, information in medical questionnaires filled out by patients at the time of consultation, and information included in video data including the patient, and to automatically make the areas high image quality. As a result, it is possible to clearly display the video of the area that the doctor wants to visually examine, thereby improving medical services.

In addition, as compared with a case where all video data is transmitted without being compressed or a case where all video data is compressed and transmitted at a constant compression ratio, it is possible to check the area to be visually examined with good image quality while preventing an increase in traffic of the network N.

Note that the example illustrated in FIG. 3 illustrates an example in which the information processing system 100 includes the information processing device 1, but the present invention is not limited to this configuration. For example, the information processing device 1 can be realized as a server installed in a data center or the like. The video data including the patient from the patient-side terminal 20 is transmitted to the server, where the server executes the processing of generating the image quality change instructions described above. The image quality change instruction from the server is sent to the patient-side terminal 20, and this allows the patient-side terminal 20 to transmit video data to the doctor-side terminal 10 in which the image quality in the change area is higher than that of other areas.

Note that each functional block that performs various types of processing described in the drawings can be configured by a processor, a memory, or another circuit in terms of hardware. In addition, the above-described processing can be realized by causing a processor to execute a program. Therefore, these functional blocks can be realized in various forms by hardware alone, software alone, or a combination thereof, and are not limited to any of them.

The programs described above can be stored and supplied to computers using various types of non-transitory computer-readable media. The non-transitory computer-readable media include various types of tangible storage media. Examples of the non-transitory computer-readable medium include a magnetic recording medium (for example, a flexible disk, a magnetic tape, or a hard disk drive), an optical magnetic recording medium (for example, a magneto-optical disk), a compact disc-read only memory (CD-ROM), a CD-R, a CD-R/W, and a semiconductor memory (for example, a mask ROM, a programmable ROM (PROM), an erasable PROM (EPROM), a flash ROM, or a random access memory (RAM). The program may be supplied to the computer by various types of transitory computer-readable media. Examples of the transitory computer-readable media include electrical signals, optical signals, and electromagnetic waves. The transitory computer-readable media can provide the program to the computer via a wired communication line such as an electric wire and optical fibers or a wireless communication line.

Although the present disclosure has been described with reference to the example embodiments, the present disclosure is not limited to the example embodiments described above. Various modifications that can be understood by those skilled in the art can be made to the configuration and details of the present disclosure within the scope of the present disclosure.

Furthermore, part or the entirety of the example embodiments described above can also be described as described in the following supplementary notes, but is not limited to the following.

### (Supplementary Note 1)

An information processing system including:
an acquisition means for acquiring video data including a subject receiving a medical service and subject-related information related to the subject included in the video data;
an identification means for identifying a change area where image quality in the video data is changed based on the subject-related information; and
an image quality changing means for changing the image quality in the identified change area.

### (Supplementary Note 2)

The information processing system according to Supplementary note 1, which further includes:
an output means for outputting a plurality of candidates for the change area based on the subject-related information, and
an accepting means for accepting a selection of at least one candidate from the plurality of candidates,
in which the image quality changing means is configured to change image quality in an area corresponding to the selected candidate.

### (Supplementary Note 3)

The information processing system according to Supplementary note 1 or 2, in which the subject-related information is at least one of medical record information of the subject, medical interview information of the subject, voice information of the subject included in the video data, or motion information of the subject included in the video data.

### (Supplementary Note 4)

The information processing system according to Supplementary note 3,
in which the acquisition means is configured to further acquire voice data of a provider of a medical service, and the identification means is configured to identify the change area based on at least one of the subject-related information or the voice data.

### (Supplementary Note 5)

The information processing system according to Supplementary note 4, in which when the voice information of the subject and the voice data of the provider are acquired, the identification means is configured to give priority to the voice data of the provider and identify the area in which the image quality in the video data is changed based on the voice data.

### (Supplementary Note 6)

The information processing system according to Supplementary note 3, in which when the voice information and the motion information of the subject are acquired, the identification means is configured to identify the change area based on both the voice information and the motion information.

### (Supplementary Note 7)

The information processing system according to Supplementary note 3, which further includes an input means that imitates a human body, and
the subject-related information includes specified position information on the human body inputted from the input means.

### (Supplementary Note 8)

The information processing system according to any one of Supplementary notes 1 to 7, which further includes a first terminal and a second terminal configured to communicate bidirectionally,
the first terminal includes a video shooting means configured to shoot the subject and generating the video data, and
the second terminal includes a display means configured to display a video in which the image quality in the change area has been changed in the video data generated by the first terminal.

### (Supplementary Note 9)

The information processing system according to Supplementary note 8, in which the image quality changing means is configured to control a compression ratio of the video data generated by the video shooting means to change the image quality in the change area.

### (Supplementary Note 10)

The information processing system according to Supplementary note 8, in which the first terminal further includes a notification means configured to notify the subject of the change area identified by the identification means.

### (Supplementary Note 11)

An information processing device including:
an acquisition means for acquiring video data including a subject receiving medical services and subject-related information related to the subject included in the video data;
an identification means for identifying a change area where image quality in the video data is changed based on the subject-related information; and
a control means for outputting an image quality change instruction to change the image quality in the identified change area.

### (Supplementary Note 12)

The information processing device according to Supplementary note 11, which further includes:
an output means for outputting a plurality of candidates for the change area based on the subject-related information, and
an accepting means for accepting a selection of at least one candidate from the plurality of candidates, and
in which the control means is configured to output the image quality change instruction to change the image quality in an area corresponding to the selected candidate.

### (Supplementary Note 13)

The information processing device according to Supplementary note 11 or 12, in which the subject-related information is at least one of medical record information of the subject, medical interview information of the subject, voice information of the subject included in the video data, or motion information of the subject included in the video data.

### (Supplementary Note 14)

The information processing device according to Supplementary note 13,
in which the acquisition means is configured to further acquire voice data of a provider of a medical service, and
the identification means is configured to identify the change area based on at least one of the subject-related information or the voice data.

### (Supplementary Note 15)

The information processing device according to Supplementary note 14, in which when the voice information of the subject and the voice data of the provider are acquired, the identification means is configured to give priority to the voice data of the provider and identify the area in which the image quality in the video data is changed based on the voice data.

### (Supplementary Note 16)

The information processing device according to Supplementary note 13, in which when the voice information and the motion information of the subject are acquired, the identification means is configured to identify the change area based on both the voice information and the motion information.

### (Supplementary Note 17)

The information processing device according to Supplementary note 13, which further includes an input means that imitates a human body, and the subject-related information includes specified position information on the human body inputted from the input means.

### (Supplementary Note 18)

The information processing device according to any one of claims 11 to 17,
in which the acquisition means
is configured to acquire, via a network, the video data generated by shooting the subject by an external video shooting means, and
further comprises a display means configured to display the video in which the image quality in the change area has been changed, in the video data generated by the video shooting means, in accordance with the image quality change instruction by the control means.

### (Supplementary Note 19)

The information processing device according to Supplementary note 18, in which the control means is configured to control a compression ratio of the video data generated by the video shooting means and generates the image quality change instruction to change the image quality in the change area.

### (Supplementary Note 20)

The information processing device according to Supplementary note 18, in which the control means is configured to further output a notification instruction to notify the subject of the change area identified by the identification means.

### (Supplementary Note 21)

An information processing method including:
acquiring video data including a subject receiving a medical service and subject-related information related to the subject included in the video data; identifying a change area where image quality in the video data is changed based on the subject-related information; and changing the image quality in the identified change area.

### (Supplementary Note 22)

A non-transitory computer-readable medium storing a program for causing a computer to execute:
processing of acquiring video data including a subject receiving a medical service and subject-related information related to the subject included in the video data;
processing of identifying a change area where image quality in the video data is changed based on the subject-related information; and
processing of changing the image quality in the identified change area.

### REFERENCE SIGNS LIST

1 INFORMATION PROCESSING DEVICE
2 ACQUISITION UNIT
3 IDENTIFICATION UNIT
4 CONTROL UNIT
5 ARITHMETIC DEVICE
6 IMAGE QUALITY CHANGE PROCESSING UNIT
10 DOCTOR-SIDE TERMINAL
11 DISPLAY DEVICE
12 VIDEO SHOOTING DEVICE
13 STORAGE DEVICE
14 INPUT DEVICE
15 COMMUNICATION DEVICE
16 VOICE INPUT DEVICE
17 VOICE OUTPUT DEVICE
18 BUS
20 PATIENT-SIDE TERMINAL
21 DISPLAY DEVICE
22 VIDEO SHOOTING DEVICE
23 STORAGE DEVICE
24 INPUT DEVICE
25 COMMUNICATION DEVICE
26 VOICE INPUT DEVICE
27 VOICE OUTPUT DEVICE
28 BUS
100 INFORMATION PROCESSING SYSTEM
101 ACQUISITION MEANS
102 IDENTIFICATION MEANS
103 IMAGE QUALITY CHANGING MEANS
111 VIDEO DISPLAY AREA
112 INFORMATION DISPLAY AREA
113 INPUT INFORMATION DISPLAY AREA
211 VIDEO DISPLAY AREA
212 INFORMATION DISPLAY AREA
213 INPUT INFORMATION DISPLAY AREA
N NETWORK

What is claimed is:

1. An information processing system comprising:
at least one memory storing instructions, and
at least one processor configured to execute the instructions to;
acquire video data including a subject receiving a medical service and subject-related information related to the subject included in the video data;
identify a change area where image quality in the video data is changed based on the subject-related information; and
change the image quality in the identified change area.

2. The information processing system according to claim 1, wherein at least one processor is further configured to execute the instructions to:
output a plurality of candidates for the change area based on the subject-related information, and
accept a selection of at least one candidate from the plurality of candidates,
wherein the image quality in an area corresponding to the selected candidate is changed.

3. The information processing system according to claim 1, wherein the subject-related information is at least one of medical record information of the subject, medical interview information of the subject, voice information of the subject included in the video data, or motion information of the subject included in the video data.

4. The information processing system according to claim 3, wherein at least one processor configured to execute the instructions to;
acquire voice data of a provider of a medical service, and
identify the change area based on at least one of the subject-related information or the voice data.

5. The information processing system according to claim 4, wherein, when the voice information of the subject and the voice data of the provider are acquired, priority is given to the voice data of the provider and the change area is identified based on the voice data.

6. The information processing system according to claim 3, wherein, when the voice information and the motion information of the subject are acquired, the change area is identified based on both the voice information and the motion information.

7. The information processing system according to claim 1, further comprising an input device that imitates a human body,
wherein the subject-related information includes specified position information on the human body inputted from the input device.

8. The information processing system according to claim 1, further comprising a first terminal and a second terminal configured to communicate bidirectionally,
wherein the first terminal configured to execute the instructions to image the subject and generating the video data, and
the second terminal configured to execute the instructions to display a video wherein the image quality in the change area has been changed in the video data generated by the first terminal.

9. The information processing system according to claim 8, wherein a compression ratio of the generated video data is controlled to change the image quality in the change area.

10. The information processing system according to claim 8, wherein the first terminal further configured to execute the instructions to notify the identified subject of the change area.

11. An information processing device comprising:
at least one memory storing instructions, and
at least one processor configured to execute the instructions to;
acquire video data including a subject receiving a medical service and subject-related information related to the subject included in the video data;
identify a change area where image quality in the video data is changed based on the subject-related information; and
output an image quality change instruction to change the image quality in the identified change area.

12. The information processing device according to claim 11, wherein at least one processor configured to execute the instructions to;
output a plurality of candidates for the change area based on the subject-related information, and
accept a selection of at least one candidate from the plurality of candidates,
wherein the image quality change instruction is output to change the image quality in an area corresponding to the selected candidate.

13. The information processing device according to claim 11, wherein the subject-related information is at least one of medical record information of the subject, medical interview information of the subject, voice information of the subject included in the video data, or motion information of the subject included in the video data.

14. The information processing device according to claim 13, wherein at least one processor configured to execute the instructions to;
acquire voice data of a provider of a medical service, and
identify the change area based on at least one of the subject-related information or the voice data.

15. The information processing device according to claim 14, wherein, when the voice information of the subject and the voice data of the provider are acquired, priority is given to the voice data of the provider and the change area is identified based on the voice data.

16. The information processing device according to claim 13, wherein, when the voice information and the motion information of the subject are acquired, the change area is identified based on both the voice information and the motion information.

17. The information processing device according to claim 11, further comprising an input device that imitates a human body,
wherein the subject-related information includes specified position information on the human body inputted from the input device.

18. The information processing device according to claim 11, wherein at least one processor configured to execute the instructions to;
acquire, via a network, the video data generated by shooting the subject by an external video shooting device, and
display the video in which the image quality in the change area has been changed, in the video data generated by the video shooting device, in accordance with the image quality change instruction.

19. The information processing device according to claim 18, wherein at least one processor configured to execute the instructions to;
control a compression ratio of the video data generated by the video shooting device and generate the image quality change instruction to change the image quality in the change area.

20-21. (canceled)

22. A non-transitory computer-readable medium storing a program for causing a computer to execute:
processing of acquiring video data including a subject receiving a medical service and subject-related information related to the subject included in the video data;
processing of identifying a change area where image quality in the video data is changed based on the subject-related information; and
processing of changing the image quality in the identified change area.
